(19) United States
(12) Patent Application Publication
(10) Pub. No.: US 2024/0374753 A1
(43) Pub. Date: Nov. 14, 2024

(54) USE OF AN AGONIST OF FORMYL PEPTIDE RECEPTORS 1 AND 2 FOR TREATING OCULAR INFLAMMATORY DISEASES

(71) Applicant: ALLGENESIS BIOTHERAPEUTICS INC., Taipei City (TW)

(72) Inventors: Ching-Yu LAI, Taipei City (TW); Tan NGUYEN, Taipei City (TW); Madhu CHERUKURY, Taipei City (TW); Sunil PATEL, Taipei City (TW)

(73) Assignee: ALLGENESIS BIOTHERAPEUTICS INC., Taipei City (TW)

(21) Appl. No.: 18/661,074

(22) Filed: May 10, 2024

Related U.S. Application Data
(60) Provisional application No. 63/465,766, filed on May 11, 2023.

(51) Int. Cl. A61K 47/69; A61K 9/00; A61K 31/198; A61K 45/06; A61P 27/02

(52) U.S. Cl. CPC A61K 47/6951; A61K 9/0048; A61K 31/198; A61K 45/06; A61P 27/02

(57) ABSTRACT

Pharmaceutical compositions containing AG-80308 and cyclodextrin are provided. Also provided are methods for treating ocular inflammatory diseases in a subject in need of such treatment by administering pharmaceutical compositions containing AG-80308 and cyclodextrin. Compositions may be formulated for ophthalmic application, for example, for ocular delivery, such as eye drops, to subjects in need thereof.

### CROSS-REFERENCE TO RELATED APPLICATION

This application claims priority from U.S. Provisional Application No. 63/465,766, filed on May 11, 2023, the disclosure of which is incorporated herein by reference in its entirety.

### FIELD

The present disclosure relates to a method for treating ocular inflammatory diseases in a subject in need of such treatment, which comprises administering a pharmaceutical composition comprising a therapeutically effective amount of AG-80308, an agonist of formyl peptide receptors 1 and 2.

### BACKGROUND

Under nonpathologic conditions, inflammatory responses are self-limiting and typically do not cause significant tissue damage. However, excessive or dysregulated inflammatory and immune processes can lead to persistent tissue damage, fibrosis, and functional impairment. The body can naturally down-regulate inflammatory responses through resolution pathways, which are mediated by classes of endogenous bioactive molecules including resolvins, lipoxins, and annexins.

The formyl peptide receptors 1 and 2 (FPR1 and FPR2) are G protein-coupled receptors (GPCRs) that play a central role in endogenous inflammation resolution pathways. Lipoxin A4 (LxA4), resolvin D1 (RvD1), and glucocorticoid-modulated protein annexin A1 (AnxA1) are key mediators of the pro-resolution pathway and all exhibit anti-inflammatory properties through selective binding to FPR1 and FPR2. These endogenous lipid ligands as well as synthetic agonists of FPR1 and FPR2 are known to control neutrophil activation and chemotaxis, macrophage phagocytosis and chemotaxis, and epithelial healing. Triggering resolution pathways with topically administered agonist molecules represents a novel approach for controlling acute and/or chronic ocular inflammation. Acute inflammatory responses are common following surgical interventions such as cataract extractions, while both acute and chronic inflammatory responses are often associated with ocular surface conditions such as dry eye disease (DED).

AG-80308 is {[(2S)-2-{[(4-bromophenyl) carbamoyl]amino}-4-methylpentanoyl]amino}acetic acid, an amide derivative of an N-substituted amino acid, whose structure is shown below:

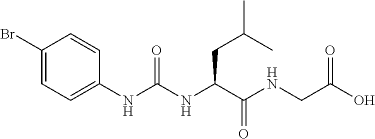 [SMILES: CC(C)C[C@H](NC(=O)Nc1ccc(Br)cc1)C(=O)NCC(=O)O]

AG-80308 and methods for making and testing the same is disclosed in U.S. Pat. Nos. 8,993,780, 8,658,803, as well as related patents and applications, the disclosures of which are incorporated herein by reference.

AG-80308 is a first-in-class small molecule agonist of the FPR1 and FPR2 receptors that in preclinical studies has demonstrated a wide margin of safety and tolerability, potency, and rapid efficacy similar to steroids without any impairment of wound healing. Thus, the FPRs represent attractive therapeutic targets for controlling inflammation in a manner that may avoid the safety concerns associated with existing classes of anti-inflammatory drugs. AG-80308 may provide a useful alternative to corticosteroids and immunosuppressive agents in treating inflammation in dry eye patients and other ocular inflammatory diseases.

### SUMMARY

According to an aspect of the disclosure, compositions are provided containing AG-80308 and a compound of the cyclodextrin family. In embodiments, pharmaceutical compositions may contain from 0.001% (w/v) to 10% (w/v) AG-80308 and from 0.1% to 10% (w/v) of a compound of the cyclodextrin family. In some embodiments, pharmaceutical compositions contains from 0.001% (w/v) to 10% (w/v) AG-80308 and from 0.1% to 0.5% (w/v) of a member of the cyclodextrin family; from 0.02% (w/v) to 0.04% (w/v) AG-80308 and from 0.2% to 0.4% (w/v) of a member of the cyclodextrin family; or 0.03% (w/v) AG-80308 and 0.3% (w/v) of a member of the cyclodextrin family. In some embodiments, the pharmaceutical composition contains 0.001% (w/v), 0.002% (w/v), 0.003% (w/v), 0.005% (w/v), 0.01% (w/v), 0.02% (w/v), or 0.03% (w/v) AG-80308 and 0.01% (w/v), 0.02% (w/v), 0.03%, (w/v) 0.05% (w/v), 0.1% (w/v), 0.2% (w/v), or 0.3% (w/v) of a member of the cyclodextrin family. In some embodiments, the pharmaceutical composition contains AG-80308 and cyclodextrin in the amounts selected from: 0.002% (w/v) AG-80308 and 0.02% (w/v) cyclodextrin; 0.002% (w/v) AG-80308 and 0.2% (w/v) cyclodextrin; 0.006% (w/v) AG-80308 and 0.06% (w/v) cyclodextrin; 0.006% (w/v) AG-80308 and 0.2% (w/v) cyclodextrin; 0.02% (w/v) AG-80308 and 0.2% (w/v) cyclodextrin; and 0.03% (w/v) AG-80308 and 0.3% (w/v) cyclodextrin.

In some embodiments, the pharmaceutical composition is formulated for ophthalmic applications, such as for topical ocular delivery, for example, by eye drops, intravitreal, intraocular, juxtascleral, subconjunctival, intracameral, or retrobulbar delivery.

In some embodiments, cyclodextrin in the pharmaceutical composition is 2-hydroxypropyl-beta-cyclodextrin (HPβCD).

In some embodiments, the pharmaceutical composition further contains citric acid monohydrate and/or sodium phosphate dibasic heptahydrate as a buffer.

In some embodiments, the buffer in the pharmaceutical composition is citric acid monohydrate at a concentration of from 0.01% to 1% (w/v), and/or sodium phosphate dibasic heptahydrate at a concentration of from of 0.01 to 1% (w/v).

In some embodiments, the pharmaceutical compositions have a pH of between 4.5 and 8.0. In some embodiments, the pharmaceutical compositions have a physiological pH.

According to an aspect of the disclosure, methods for treating ocular inflammatory diseases with the pharmaceutical compositions of the present disclosure in a subject in need thereof are provided. In embodiments, compositions may be administered by topical ocular delivery, for example, by eye drops, intravitreal, intraocular, juxtascleral, subconjunctival, intracameral, or retrobulbar delivery.

Compositions formulated as eye drops may be administered, for example, one to ten times per day in a total volume of approximately 30 μl per drop. In embodiments, compositions may be administered one to six times per a day, one to five times per day, one to four times per day, one to three times per day, one to two times per day, once per day, twice per day, three times per day, four times per day, five times per day or six time per day. The compositions may be administered, for example, from 1 to 5 drops, 1 to 4 drops, 1 to 3 drops, 1 to 2 drops, 1 drop, 2 drops, 3 drops, 4 drops, 5 drops, or 6 drops per administration in affected eye. In some embodiments, one drop of a pharmaceutical composition of the present disclosure is administered per eye 1 to 6 times a day.

Ocular inflammatory diseases that may be treated with the compositions of the present disclosure include, but are not limited to, a dry eye disease, uveitis, keratitis, choroiditis, episcleritis, sarcoidosis, scleritis, ocular cicatricial pemphigoid, orbital inflammatory syndrome, ocular graft-vs.-host disease (GvHD), conjunctivitis, allergic eye disease, post-surgical corneal inflammation, blepharitis, MGD, and others. The compositions may be administered in combination with one or more other drugs including, but not limited to, a steroid and a non-steroidal anti-inflammatory drug, antihistamine, immunosuppressive, artificial tears, mucin secretagogue, peptide regenerative medicines, reactive aldehyde species (RASP) inhibitors, cholinergic agonists, and anti-evaporative treatments. The compositions may be administered concurrently or sequentially with other drugs.

### BRIEF DESCRIPTION OF THE DRAWINGS

Further features, the nature, and various advantages of the disclosed subject matter will be more apparent from the following detailed description and the accompanying drawings in which:

FIG. 1 is a graph depicting Total Corneal Staining Score in subjects with dry eye disease treated with 0.001%, 0.03%, or 0.1% AG-80308 in 10% cyclodextrin (Formulation A) or 0.03% AG-80308 in 0.3% cyclodextrin (Formulation B) using the NEI Scale at the specified time points.

FIG. 2 is a graph depicting Central Corneal Staining Score in subjects with dry eye disease treated with 0.001%, 0.03%, or 0.1% AG-80308 in 10% cyclodextrin (Formulation A) or 0.03% AG-80308 in 0.3% cyclodextrin (Formulation B) using the NEI Scale at the specified time points.

FIG. 3 is a graph depicting Eye Discomfort Score in subjects with dry eye disease treated with 0.001%, 0.03%, or 0.1% AG-80308 in 10% cyclodextrin (Formulation A) or 0.03% AG-80308 in 0.3% cyclodextrin (Formulation B) using the visual analogue scale (VAS) questionnaire at the specified time points.

FIG. 4 is a graph depicting Eye Dryness Score in subjects with dry eye disease treated with 0.001%, 0.03%, or 0.1% AG-80308 in 10% cyclodextrin (Formulation A) or 0.03% AG-80308 in 0.3% cyclodextrin (Formulation B) using the VAS questionnaire at the specified time points.

### DETAILED DESCRIPTION

The following illustrates certain features of the disclosure and is not meant to limit the scope of the disclosure in any way. The failure to discuss a specific feature or embodiment of the disclosure, or the inclusion of one or more features in this summary section, should not be construed to limit any claims.

It is also to be understood that the disclosure is not limited to particular compositions, methods, and experimental conditions described, as such compositions, methods, and conditions may vary. It is also to be understood that the terminology used herein is for purposes of describing particular embodiments only, and is not intended to be limiting, since scope will be limited only in the appended claims.

As used in this specification and the appended claims, the singular forms “a”, “an”, and “the” include plural references unless the context clearly dictates otherwise. Thus, for example, references to “an excipient” includes one excipient known in the art which will become apparent to those persons skilled in the art upon reading this disclosure and so forth.

Unless defined otherwise, all technical and scientific terms used herein have the same meaning as commonly understood by persons skilled in the art to which this disclosure belongs. Any methods and materials similar or equivalent to those described herein may be used in the practice or testing of the disclosure, as it will be understood that modifications and variations are encompassed within the spirit and scope of the instant disclosure.

Any range will be understood to encompass and be a disclosure of each discrete point and subrange within the range. Stated differently, the ranges in the present disclosure are equivalent to a subset of the unwieldy and lengthy description of every possible combination of these discrete values, presented in an easily understood shorthand format (i.e., a range). The upper or lower value of any numerical range may also be replaced with the upper or lower value of another numerical range, respectively. The upper and lower value of a numerical range may also be replaced with the upper and lower value described in Examples below.

As used herein, “about,” “approximately,” “substantially,” and “significantly” will be understood by persons skilled in the art and will vary in some extent depending on the context in which they are used. If there are uses of the term which are not clear to persons skilled in the art given the context in which it is used, “about” and “approximately” will mean plus or minus ≤10% of particular value inclusive of the value, and “substantially” and “significantly” will mean plus or minus ≥10% of the particular value inclusive of the value. “Comprising,” “consisting essentially of,” and “consisting of” have their customary meaning in the art.

Reference throughout this specification to “one embodiment,” “an embodiment,” or similar language means that a particular feature, structure, or characteristic described in connection with the indicated embodiment is included in at least one embodiment of the present solution. Thus, the phrases “in one embodiment”, “in an embodiment,” and similar language throughout this specification may, but do not necessarily, all refer to the same embodiment.

Furthermore, the described features, advantages, and characteristics of the present disclosure may be combined in any suitable manner in one or more embodiments. Persons skilled in the relevant art will recognize, in light of the description herein, that the present disclosure may be practiced without one or more of the specific features or advantages of a particular embodiment. In other instances, additional features and advantages may be recognized in certain embodiments that may not be present in all embodiments of the present disclosure.

Provided herein is a compound referred to as AG-80308 having the formula C₁₅H₂₀BrN₃O₄, molecular weight of 386.24 g/mol, and formulations comprising AG-80308 or pharmaceutically acceptable salts, solvates, hydrates, crystal forms and individual isomers, enantiomers, and diastereoisomers thereof. Prodrugs and derivatives of AG-80308, for example, esters or amides of AG-80308, may be used in place of AG-80308 where appropriate as would be understood by persons skilled in the art.

In embodiments, there are provided pharmaceutical compositions including AG-80308 in a pharmaceutically acceptable carrier or vehicle. The phrase “pharmaceutically acceptable” means the carrier, diluent or excipient must be compatible with the other ingredients of the formulation and not deleterious to the recipient thereof. In some embodiments, the pharmaceutical compositions may include additional active ingredients or inactive ingredients. In some embodiments the compositions are formulated for ocular delivery such as eye drops.

Pharmaceutical compositions according to the embodiments may be used in the form of a solid, a solution, an emulsion, a dispersion, such as a suspension, a patch, a micelle, a liposome, and the like, wherein the resulting composition contains one or more compounds of the present disclosure, as an active ingredient, in admixture with an organic or inorganic carrier or excipient suitable for enteral or parenteral applications.

Pharmaceutical compositions according to the embodiments may be administered by any suitable route. Such routes may include, but are not limited to, transdermal, parenteral, subcutaneous, intranasal, via an implant stent, intrathecal, intravitreal, topical to the eye, to the back of the eye, intramuscular, intravenous, intrarectal, suprachoroidal, sub-tenon, or as implants. Additionally, the formulations may be designed to delay release of the active compound over a given period of time, or to carefully control the amount of drug released at a given time during the course of therapy, when administered by such routes as intravitreal, suprachoroidal, sub-tenon, implants, etc.

In embodiments, AG-80308 may be combined, for example, with the acceptable non-toxic, pharmaceutically acceptable carriers for tablets, pellets, capsules, suppositories, solutions, emulsions, suspensions, and any other form suitable for use. The carriers which can be used include glucose, lactose, gum acacia, gelatin, mannitol, starch paste, magnesium trisilicate, talc, corn starch, keratin, colloidal silica, potato starch, urea, medium chain triglycerides, dextrans, and other carriers suitable for use in manufacturing preparations, in solid, semisolid, or liquid form. In addition, auxiliary, stabilizing, thickening and coloring agents and perfumes may be used. AG-80308 are included in the pharmaceutical composition in an amount sufficient to produce the desired effect upon the process or disease condition.

In some embodiments, compositions may be formulated for ophthalmic use. Pharmaceutical compositions for ophthalmic may be in a form suitable for topical use, such as eyedrops, for example, as oily suspensions, as aqueous or nonaqueous solutions or suspensions, or as oil-in-water or water-in-oil liquid emulsions.

Pharmaceutical compositions for ophthalmic use may be prepared by combining a therapeutically effective amount of AG-80308 or a pharmaceutically acceptable salt or other form thereof as an active ingredient with ophthalmically acceptable pharmaceutical excipients and by preparation of unit dosage suitable for ophthalmic use. The therapeutically effective amount typically is between about 0.001 and about 5% (w/v), preferably about 0.001 to about 2.0% (w/v), more preferably 0.001 to 0.05% (w/v), or 0.03% (w/v) in liquid formulations.

When the pharmaceutical compositions are administered as implants,

For ophthalmic application, solutions are preferably prepared using a physiological saline solution as a main vehicle. The pH of such ophthalmic solutions should preferably be maintained between 4.5 and 8.0 with an appropriate buffer system if needed. A pH near that of human tears being preferred but not essential. The formulations may also contain conventional pharmaceutically acceptable excipients, including but not limited to preservatives, osmotic adjusting agents, polymers, stabilizers, and surfactants. Preferred preservatives that may be used in the pharmaceutical compositions of the present disclosure include, but are not limited to, benzalkonium chloride, stabilized oxychloro complexes, sorbic acid, and polyquaternium compounds. Preferred surfactants include but are not limited to polyoxyethylene (POE) castor oil (Cremophor) and hydrogenated castor oil derivatives, POE sorbitan fatty acid esters, sorbitan fatty acid esters, such as polysorbate 80, poloxamers, tyloxapol, POE fatty acid esters and ethers, POE phenoxy alkyl ethers, and sodium dodecyl sulfate, among others. Likewise, various preferred vehicles may be used in the ophthalmic preparations of the present disclosure. These vehicles include additives such as, but are not limited to, polyvinyl alcohol, povidone, hydroxypropyl methyl cellulose, poloxamers, carboxymethyl cellulose, hydroxyethyl cellulose, cyclodextrins, and purified water.

Tonicity adjustors may be added as needed or convenient. They include, but are not limited to ionic compounds, particularly sodium chloride, potassium chloride, and calcium chloride or nonionic compounds such as mannitol, propylene glycol, PEGs, sorbitol, trehalose, and glycerin, or any other suitable ophthalmically acceptable tonicity adjustor.

Various buffers and means for adjusting pH may be used so long as the resulting preparation is ophthalmically acceptable. Accordingly, buffer components include but are not limited to acetic acid and salts, citric acid and salts, phosphoric acid and salts, boric acid and salts, sodium dihydrogen phosphate, disodium hydrogen phosphate, and others. Acids or bases may be used to adjust the pH of these formulations as needed and may include but are not limited to HCl, sulfuric acid, nitric acid, NaOH, KOH, tromethamine, and trolamine. In embodiments, citric acid monohydrate, sodium phosphate dibasic heptahydrate may be used as components of a buffer. The buffer, if used in the formulation, is at concentration of from 1 mM to 100 mM, preferably 5 mM to 50 mM. [ . . . ]

In a similar manner, an ophthalmically acceptable antioxidant for use in the present disclosure includes, but is not limited to, sodium metabisulfite, sodium thiosulfate, and acetylcysteine.

Other excipient components which may be included in the ophthalmic preparations are chelating agents. The preferred chelating agent is edetate disodium, although other chelating agents may also be used in place of or in combination with it.

The ophthalmic formulations of the present disclosure may be conveniently packaged in forms suitable for metered application, such as in containers equipped with a dropper or dropper tip, to facilitate application to the eye. Containers suitable for dropwise application are usually made of suitable inert, non-toxic plastic material. The volume of eye drops in a container may be between about 0.1 and about 15 ml. Eye drops may be provided in a container containing one or more doses (units of use). A unit of use container may contain sufficient volume of formulation to provide from about two to about 10 drops. Preservative-free solutions are often formulated in non-resealable containers containing up to about ten, preferably up to about five units doses, where a typical unit dose is from one to about 8 drops, preferably one to about 3 drops. The volume of one drop usually is about 20-35 μL. When formulations are provided in containers containing multiple units of use, the formulation must be protected from microbial contamination either through use of an effective concentration of an antimicrobial preservative or system, packaging capable of preventing microbial contamination, or demonstration that the formulation is self-preserving. Containers containing a single or multiple units of use may be packaged into packages containing multiple containers. Such packages may contain, for example, a number of dosage units suitable for up to about one month's therapy.

The pharmaceutical compositions may be in the form of a sterile injectable solutions, suspension or emulsions, using excipients and methods known to persons skilled in the art. This suspension may be formulated according to known methods using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate or the like. Buffers, preservatives, antioxidants, and the like can be incorporated as required.

Pharmaceutical compositions containing AG-80308 may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of a sweetening agent such as sucrose, lactose, or saccharin, flavoring agents such as peppermint, oil of wintergreen or cherry, coloring agents and preserving agents in order to provide pharmaceutically elegant and palatable preparations. Tablets containing AG-80308 in admixture with non-toxic pharmaceutically acceptable excipients may also be manufactured by known methods. The excipients used may be, for example, (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

In some cases, formulations for oral use may be in the form of hard gelatin capsules wherein AG-80308 is mixed with an inert solid diluent, for example, calcium carbonate, calcium phosphate or kaolin. They may also be in the form of soft gelatin capsules wherein AG-80308 is mixed with water or an oil medium, for example, peanut oil, liquid paraffin or olive oil.

Formulations for ophthalmic use may contain AG-80308 in an amount, for example, from 0.001% (w/v) to 10% (w/v), from 0.001% (w/v) to 9% (w/v), from 0.001% (w/v) to 8% (w/v), from 0.001% (w/v) to 7% (w/v), from 0.001% (w/v) to 6% (w/v), from 0.001% (w/v) to 5% (w/v), from 0.001% (w/v) to 4% (w/v), from 0.001% (w/v) to 3% (w/v), from 0.001% (w/v) to 2% (w/v), from 0.001% (w/v) to 1% (w/v), from 0.001% (w/v) to 0.9% (w/v), from 0.001% (w/v) to 0.8% (w/v), from 0.001% (w/v) to 0.7% (w/v), from 0.001% (w/v) to 0.6% (w/v), from 0.001% (w/v) to 0.5% (w/v), from 0.001% (w/v) to 0.4% (w/v). from 0.001% (w/v) to 0.3% (w/v), from 0.001% (w/v) to 0.2% (w/v), from 0.001% (w/v) to 0.1% (w/v), from 0.001% (w/v) to 0.09% (w/v), from 0.001% (w/v) to 0.08% (w/v), from 0.001% (w/v) to 0.07% (w/v), from 0.001% (w/v) to 0.06% (w/v), from 0.001% (w/v) to 0.05% (w/v), from 0.001% (w/v) to 0.04% (w/v), from 0.001% (w/v) to 0.03% (w/v), from 0.001% (w/v) to 0.02% (w/v), from 0.001% (w/v) to 0.01% (w/v), from 0.001% (w/v) to 0.009% (w/v), from 0.001% (w/v) to 0.008% (w/v), from 0.001% (w/v) to 0.007% (w/v), from 0.001% (w/v) to 0.006% (w/v), from 0.001% (w/v) to 0.005% (w/v), from 0.001% (w/v) to 0.004% (w/v), from 0.001 (w/v) to 0.003% (w/v), or from 0.001 (w/v) to 0.002% (w/v).

Formulations for topical ophthalmic use may contain AG-80308 in an amount, for example, from 0.002% (w/v) to 1% (w/v), from 0.003% (w/v) to 1% (w/v), from 0.004% (w/v) to 1% (w/v), from 0.005% (w/v) to 1% (w/v), from 0.006% (w/v) to 1% (w/v), from 0.007% (w/v) to 1% (w/v), from 0.008% (w/v) to 1% (w/v), from 0.009% (w/v) to 1% (w/v), from 0.01% (w/v) to 1% (w/v), from 0.02% (w/v) to 1% (w/v), from 0.03% (w/v) to 1% (w/v), from 0.04% (w/v) to 1% (w/v), from 0.05% (w/v) to 1% (w/v), from 0.06% (w/v) to 1% (w/v), from 0.07% (w/v) to 1% (w/v), from 0.08% (w/v) to 1% (w/v), from 0.09% (w/v) to 1% (w/v), from 0.1% (w/v) to 1% (w/v), from 0.2% (w/v) to 1% (w/v), from 0.3% (w/v) to 1% (w/v), from 0.4% (w/v) to 1% (w/v), from 0.5% (w/v) to 1% (w/v), from 0.6% (w/v) to 1% (w/v), from 0.7% (w/v) to 1% (w/v), from 0.8% (w/v) to 1% (w/v), or from 0.9% (w/v) to 1% (w/v).

In embodiments, formulations may contain AG-80308 in an amount for example, from 0.002% (w/v) to 0.1% (w/v), from 0.003% (w/v) to 0.09% (w/v), from 0.004% (w/v) to 0.08% (w/v), from 0.005% (w/v) to 0.07% (w/v), from 0.006% (w/v) to 0.06% (w/v), from 0.007% (w/v) to 0.05% (w/v), from 0.008% (w/v) to 0.04% (w/v), from 0.009% (w/v) to 0.03% (w/v), from 0.01% (w/v) to 0.03% (w/v), or from 0.02% (w/v) to 0.03% (w/v).

In embodiments, formulations may contain AG-80308 in an amount, for example, of 0.001% (w/v), 0.002% (w/v), 0.003% (w/v), 0.004% (w/v), 0.005% (w/v), 0.006% (w/v), 0.007% (w/v), 0.008% (w/v), 0.009% (w/v), 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.1% (w/v), 0.2% (w/v), 0.3% (w/v), 0.4% (w/v), 0.5% (w/v), 0.6% (w/v), 0.7% (w/v), 0.8% (w/v), 0.9% (w/v), or 1% (w/v).

Cyclodextrins are a family of cyclic oligosaccharides containing a macrocyclic ring of glucose subunits joined by α-1,4 glycosidic bonds. Typical cyclodextrins contain a number of glucose monomers ranging from six to eight units in a ring. The largest well-characterized cyclodextrin contains 32 1,4-anhydroglucopyranoside units. Poorly characterized mixtures, containing about 150-membered cyclic oligosaccharides are also known. Cyclodextrins may be obtained from biodegradation of starch using glucanotransferase enzyme. Typically, cyclodextrins have three forms: α-cyclodextrin, β-cyclodextrin, and γ-cyclodextrin. They are used in food, pharmaceutical formulations, e.g., for drug delivery, chemical industries, as well as agriculture and environmental engineering. Cyclodextrins may be modified or unmodified. Modified cyclodextrins include, for example, hydroxypropyl-beta-cyclodextrin (HPβCD), methylated cyclodextrins, sulfobutyl ether-beta-cyclodextrin (SBEβCD), carboxymethyl-beta-cyclodextrin (CMβCD) and polymer-modified cyclodextrins. Unless specified otherwise, cyclodextrin and cyclodextrins as used herein include any modified or unmodified compounds of the cyclodextrin family and mixtures thereof.

In pharmaceutical formulations cyclodextrins may be used to improve solubility of drugs, as carriers or excipients and for other purposes. Some of the advantages of using cyclodextrins are their ability to enhance solubility, dissolution, and bioavailability of poorly soluble drugs; ability to enhance drug stability; ability to modify the drug release site and/or time profile; and ability to reduce drug side effects (for example, gastric or ocular irritation). An important property of cyclodextrins for use as drug carriers is that their inner cavity is hydrophobic, while their surface is hydrophilic. This enables cyclodextrins to form complexes with lipophilic molecules. In pharmaceutical formulations, cyclodextrins can be used to encapsulate active ingredients since they can host active molecules in their internal hydrophobic cavities. Further cyclodextrins have excellent biocompatibility, safety, and stability, making them suitable for various drug delivery systems including for ophthalmic use, such as for topical ophthalmic delivery.

Cyclodextrin has emerged as a promising ingredient in ocular formulations due to its ability to enhance the solubility and stability of drugs intended for ophthalmic delivery. These cyclic oligosaccharides form inclusion complexes with hydrophobic drugs, thereby increasing their aqueous solubility and bioavailability. Cyclodextrin is cyclic oligosaccharides with α-(1,4) linked α-D-glucopyranose units. In nature, three types are formed by bacterial digestion of starch; α-cyclodextrin with 6, β-cyclodextrin with 7, and γ-cyclodextrin with 8 glucopyranose units. In ocular formulations, cyclodextrin can improve the therapeutic efficacy of poorly water-soluble drugs by facilitating their absorption and retention on the ocular surface. Moreover, cyclodextrin complexes can protect sensitive drugs from degradation, oxidation, and hydrolysis, thereby prolonging their shelf life and ensuring consistent drug delivery. Additionally, cyclodextrin-based formulations are well-tolerated and exhibit minimal ocular irritation, making them suitable for ocular drug delivery applications. Data also support that 2-hydroxypropyl-beta-cyclodextrin (HPβCD) can stabilize AG-80308 by increasing solubility of AG-80308 and its degradant, preventing precipitation during storage or stress conditions.

In embodiments of pharmaceutical compositions of the present disclosure, various suitable cyclodextrins and their mixtures may be used. In some embodiments, HPβCD is used, such as, for example, HPβCD available from multiple sources, including, but not limited to, Wacker Chemie AG, Cerestar USA Inc., Pharmatec Inc., Roquette Frères, Shandong Binzhou Zhiyuan BioLOGICAL Technology Co. Ltd, and Zibo Qianhui Biological Technology Co. Ltd.

Formulations for topical ophthalmic use may contain a cyclodextrin in an amount, for example, from 0.01% (w/v) to 15% (w/v), from 0.01% (w/v) to 14% (w/v), from 0.01% (w/v) to 13% (w/v), from 0.01% (w/v) to 12% (w/v), from 0.01% (w/v) to 11% (w/v), from 0.01% (w/v) to 10% (w/v), from 0.01% (w/v) to 9% (w/v), from 0.01% (w/v) to 8% (w/v), from 0.01% (w/v) to 7% (w/v), from 0.01% (w/v) to 6% (w/v), from 0.01% (w/v) to 5% (w/v), from 0.01% (w/v) to 4% (w/v), from 0.01% (w/v) to 3% (w/v), from 0.01% (w/v) to 2% (w/v), from 0.01% (w/v) to 1% (w/v), from 0.01% (w/v) to 0.9% (w/v), from 0.01% (w/v) to 0.8% (w/v), from 0.01% (w/v) to 0.7% (w/v), from 0.01% (w/v) to 0.6% (w/v), from 0.01% (w/v) to 0.5% (w/v), from 0.01% (w/v) to 0.4% (w/v), from 0.01% (w/v) to 0.3% (w/v), from 0.01% (w/v) to 0.2% (w/v), from 0.001% (w/v) to 0.1% (w/v), from 0.001% (w/v) to 0.09% (w/v), from 0.001% (w/v) to 0.08% (w/v), from 0.01% (w/v) to 0.07% (w/v), from 0.01% (w/v) to 0.06% (w/v), from 0.01% (w/v) to 0.05% (w/v), from 0.01% (w/v) to 0.04% (w/v), from 0.01% (w/v) to 0.03% (w/v), or from 0.01% (w/v) to 0.02% (w/v).

Formulations for topical ophthalmic use may contain a cyclodextrin in an amount, for example, from 0.01% (w/v) to 15% (w/v), from 0.02% (w/v) to 15% (w/v), from 0.03% (w/v) to 15% (w/v), from 0.04% (w/v) to 15% (w/v), from 0.05% (w/v) to 15% (w/v), from 0.06% (w/v) to 15% (w/v), from 0.07% (w/v) to 15% (w/v), from 0.08% (w/v) to 15% (w/v), from 0.09% (w/v) to 15% (w/v), from 0.1% (w/v) to 15% (w/v), from 0.2% (w/v) to 15% (w/v), from 0.3% (w/v) to 15% (w/v), from 0.4% (w/v) to 15% (w/v), from 0.5% (w/v) to 15% (w/v), from 0.6% (w/v) to 15% (w/v), from 0.7% (w/v) to 15% (w/v), from 0.8% (w/v) to 15% (w/v), from 0.9% (w/v) to 15% (w/v), from 1% (w/v) to 15% (w/v), from 2% (w/v) to 15% (w/v), from 3% (w/v) to 15% (w/v), from 4% (w/v) to 15% (w/v), from 5% (w/v) to 15% (w/v), from 6% (w/v) to 15% (w/v), from 7% (w/v) to 15% (w/v), from 8% (w/v) to 15% (w/v), from 9% (w/v) to 10% (w/v), from 11% (w/v) to 15% (w/v), from 12% (w/v) to 15% (w/v), from 13% (w/v) to 15% (w/v), or from 14% (w/v) to 15% (w/v).

Formulations for topical ophthalmic use may contain a cyclodextrin in an amount, for example, from 0.04% (w/v) to 10% (w/v), from 0.05% (w/v) to 10% (w/v), from 0.06% (w/v) to 10% (w/v), from 0.07% (w/v) to 10% (w/v), from 0.08% (w/v) to 10% (w/v), from 0.09% (w/v) to 10% (w/v), from 0.1% (w/v) to 10% (w/v), from 0.2% (w/v) to 10% (w/v), from 0.3% (w/v) to 10% (w/v), from 0.4% (w/v) to 10% (w/v), from 0.5% (w/v) to 10% (w/v), from 0.6% (w/v) to 10% (w/v), from 0.7% (w/v) to 10% (w/v), from 0.8% (w/v) to 10% (w/v), from 0.9% (w/v) to 10% (w/v), from 1% (w/v) to 10% (w/v), from 2% (w/v) to 10% (w/v), from 3% (w/v) to 10% (w/v), from 4% (w/v) to 10% (w/v), from 5% (w/v) to 10% (w/v), from 6% (w/v) to 10% (w/v), from 7% (w/v) to 10% (w/v), from 8% (w/v) to 10% (w/v), or from 9% (w/v) to 10% (w/v).

Formulations for topical ophthalmic use may contain a cyclodextrin in an amount, for example, from 0.02% (w/v) to 1% (w/v), from 0.03% (w/v) to 1% (w/v), from 0.04% (w/v) to 1% (w/v), from 0.05% (w/v) to 1% (w/v), from 0.06% (w/v) to 1% (w/v), from 0.07% (w/v) to 1% (w/v), from 0.08% (w/v) to 1% (w/v), from 0.09% (w/v) to 1% (w/v), from 0.1% (w/v) to 1% (w/v), from 0.2% (w/v) to 1% (w/v), from 0.3% (w/v) to 1% (w/v), from 0.4% (w/v) to 1% (w/v), from 0.5% (w/v) to 1% (w/v), from 0.6% (w/v) to 1% (w/v), from 0.7% (w/v) to 1% (w/v), from 0.8% (w/v) to 1% (w/v), or from 0.9% (w/v) to 1% (w/v).

In embodiments, formulations may contain a cyclodextrin in an amount for example, from 0.02% (w/v) to 1% (w/v), from 0.03% (w/v) to 0.9% (w/v), from 0.04% (w/v) to 0.8% (w/v), from 0.05% (w/v) to 0.7% (w/v), from 0.06% (w/v) to 0.6% (w/v), from 0.07% (w/v) to 0.5% (w/v), from 0.08% (w/v) to 0.4% (w/v), from 0.09% (w/v) to 0.3% (w/v), from 0.1% (w/v) to 0.3% (w/v), or from 0.2% (w/v) to 0.3% (w/v).

In embodiments, formulations may contain a cyclodextrin in an amount for example, 0.01% (w/v), 0.02% (w/v), 0.03% (w/v), 0.04% (w/v), 0.05% (w/v), 0.06% (w/v), 0.07% (w/v), 0.08% (w/v), 0.09% (w/v), 0.1% (w/v), 0.2% (w/v), 0.3% (w/v), 0.4% (w/v), 0.5% (w/v), 0.6% (w/v), 0.7% (w/v), 0.8% (w/v), 0.9% (w/v), 1% (w/v), 2% (w/v), 3% (w/v), 4% (w/v), 5% (w/v), 6% (w/v), 7% (w/v), 8% (w/v), 9% (w/v), 10% (w/v), 11% (w/v), 12% (w/v), 13% (w/v), 14%, or 15% (w/v).

In some embodiments, compositions may contain, for example, 0.001% (w/v), 0.03% (w/v), or 0.1% (w/v) AG-80308 in 10% (w/v) of a cyclodextrin. In some embodiments, compositions may contain, for example, 0.003% (w/v) AG-80308 in 0.3% (w/v) of a cyclodextrin.

In another aspect, provided herein is a method for treating ocular inflammatory diseases, which comprises administering a pharmaceutical composition, comprising a therapeutically effective amount AG-80308. Ocular inflammatory diseases include but not limited to, uveitis, dry eye, keratitis, ocular graft-vs.-host disease (GvHD), conjunctivitis, allergic eye disease, post-surgical corneal inflammation, blepharitis, MGD, infectious keratitis, herpetic keratitis, corneal angiogenesis, ocular cicatricial pemphigoid, and conditions affecting the posterior part of the eye, such as maculopathies and retinal degeneration including non-exudative age related macular degeneration, exudative age related macular degeneration, choroidal neovascularization, diabetic retinopathy (proliferative), retinopathy of prematurity (ROP), acute macular neuroretinopathy, central serous chorioretinopathy, cystoid macular edema, and diabetic macular edema, lymphangiogenesis, retinitis, and choroiditis such as acute multifocal placoid pigment epitheliopathy, Behcet's disease, birdshot retinochoroidopathy, infectious diseases (e.g., syphilis, lyme, tuberculosis, toxoplasmosis), intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi-and Harada syndrome; vascular diseases/exudative diseases such as retinal arterial occlusive disease, central retinal vein occlusion, cystoids macular edema, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angiitis, sickle cell retinopathy and other hemoglobinopathies, angioid streaks, familial exudative vitreoretinopathy, and Eales disease; traumatic/surgical conditions such as sympathetic ophthalmia, uveitic retinal disease, retinal detachment, trauma, post-surgical corneal wound healing, conditions caused by laser, conditions caused by photodynamic therapy, photocoagulation, hypoperfusion during surgery, radiation retinopathy, and bone marrow transplant retinopathy; proliferative disorders such as proliferative vitreal retinopathy and epiretinal membranes, and proliferative diabetic retinopathy; infectious disorders such as ocular histoplasmosis, ocular toxocariasis, presumed ocular histoplasmosis syndrome (PONS), endophthalmitis, toxoplasmosis, retinal diseases associated with HIV infection, choroidal disease associate with HIV infection, uveitic disease associate with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; and miscellaneous other diseases affecting the posterior part of the eye such as punctate inner choroidopathy, acute posterior multifocal placoid pigment epitheliopathy, myopic retinal degeneration, and acute retinal pigment epitheliitis, systemic inflammatory diseases such as stroke, coronary artery disease, obstructive airway diseases, HIV-mediated retroviral infections, cardiovascular disorders including coronary artery disease, neuroinflammation, neurological disorders, pain and immunological disorders, asthma, allergic disorders, inflammation, systemic lupus erythematosus, psoriasis, CNS disorders such as Alzheimer's disease, arthritis, sepsis, cachexia, angina pectoris, dermal wound healing, burns, rosacea, atopic dermatitis, acne, psoriasis, seborrheic dermatitis, actinic keratoses, viral warts, autoimmune diseases such as photoaging, rheumatoid arthritis and related inflammatory disorders, inflammatory bowel disease Sjogren's syndrome, alopecia, glaucoma, branch vein occlusion, Best's vitelliform macular degeneration, retinitis pigmentosa, proliferative vitreoretinopathy (PVR), and any other degenerative disease of either the photoreceptors or the RPE (Perretti, Mauro et al. Pharmacology & Therapeutics 127 (2010) 175-188).

These compositions are useful for the treatment of mammals, including humans, with a range of conditions and diseases that are alleviated by the modulation of the FPR1 and FPR2 receptors including, but not limited to the treatment of wet and dry age-related macular degeneration (ARMD), diabetic retinopathy (proliferative), retinopathy of prematurity (ROP), diabetic macular edema, uveitis, dry eye, keratitis, ocular graft-vs.-host disease (GvHD), conjunctivitis, allergic eye disease, post-surgical corneal inflammation, blepharitis, MGD, ocular cicatricial pemphigoid, retinal vein occlusion, cystoids macular edema, glaucoma, branch vein occlusion, Best's vitelliform macular degeneration, retinitis pigmentosa, proliferative vitreoretinopathy (PVR), and any other degenerative disease of either the photoreceptors or the RPE.

Such methods can be performed, for example, by administering to a subject in need thereof a therapeutically effective amount of AG-80308 or pharmaceutically acceptable salts, hydrates, solvates, crystal forms and individual isomers, enantiomers, and diastereoisomers thereof.

The actual amount of the compound to be administered in any given case will be determined by a physician taking into account the relevant circumstances, such as the severity of the condition, the age and weight of the patient, the patient's general physical condition, the cause of the condition, and the route of administration.

Since individual subjects may present a wide variation in severity of symptoms and each drug has its unique therapeutic characteristics, the precise mode of administration and dosage employed for each subject is left to the discretion of the practitioner.

The pharmaceutical compositions described herein are useful as medicaments in mammals, including humans, for treatment of diseases and/or alleviations of conditions which are responsive to treatment by agonists or functional antagonists of FPR1 and FPR2. Thus, in further embodiments of the disclosure, there are provided methods for treating a disorder associated with modulation of FPR1 and FPR2. Such methods can be performed, for example, by administering to a subject in need thereof a pharmaceutical composition containing a therapeutically effective amount of AG 80308. As used herein, the term “therapeutically effective amount” means the amount of the pharmaceutical composition that will elicit the biological or medical response of a subject in need thereof that is being sought by the researcher, veterinarian, medical doctor or other clinician. In some embodiments, the subject in need thereof is a mammal. In some embodiments, the mammal is human.

### Examples

A multicenter, double-masked, randomized, parallel-group Phase 1b study evaluating the safety, tolerability, and dose-response of AG-80308 ophthalmic solution administered twice daily for 3 months in 84 patients with dry eye disease was conducted. Patients were assigned 1:1:1:1 to receive 0.001%, 0.03%, and 0.1% AG-80308 each in 10% of a cyclodextrin (Formulation A; also referred to herein as Formulation A (0.001%), Formulation A (0.03%), and Formulation A (0.1%), respectively) or 0.03% AG-80308 in 0.3% of a cyclodextrin (Formulation B). 2-Hydroxypropyl-beta-cyclodextrin (HPβCD) was used in formulations A and B. The efficacy measures included both signs (ocular surface staining, tear film breakup time, Schirmer's test, meibomian gland assessments) and symptoms (7-item visual analog scale (VAS) assessments: burning/stinging, itching, foreign body sensation, eye discomfort, eye dryness, photophobia, pain and ocular surface disease index).

In this study, a single eye drop (approximately 30 μL) of AG-80308 was administered BID (approximately 12 hours apart) to the study eye every day until the Day 84 visit.

### Corneal Fluorescein Staining (Sign Improvement)

Corneal fluorescein staining was assessed in each eye at Visit 1 (Day −30 to Day −2), Visit 2 (Day 1), Visit 4 (Day 43), and Visit 5 (Day 84) using the NEI Scale. The NEI Scale uses a standardized grading system of 0 to 3 using whole number units for each of the three areas on each cornea. Grade 0 was specified when no staining is present. The following regions were analyzed for the study eye: inferior, superior, central, temporal, nasal and corneal sum (sum of all five regions).

### Total Corneal Staining Score

Corneal sum staining score at baseline (Day 1) was 8.8 [2.12], 7.9 [1.89], 8.6 [1.83], and 8.7 [1.62] in the group treated with Formulation A (0.001%), (0.03%), (0.1%) and Formulation B, respectively. Hereinafter, the values are expressed in the format Mean [+SE].

At Visit 4 (Day 43), all treatment groups showed improvement in the mean change from baseline. Changes from baseline were −2.1 [0.57], −1.4 [0.69], −2.1 [0.59], and −1.4 [0.59] in the group treated with Formulation A (0.001%), (0.03%), (0.1%) and Formulation B, respectively.

At Visit 5 (Day 84), similar improvements were observed. Changes from baseline were −2.7 [0.57], −1.8 [0.69], −2.3 [0.60], and −1.9 [0.59] in the group treated with Formulation A (0.001%), (0.03%), (0.1%) and Formulation B, respectively, respectively.

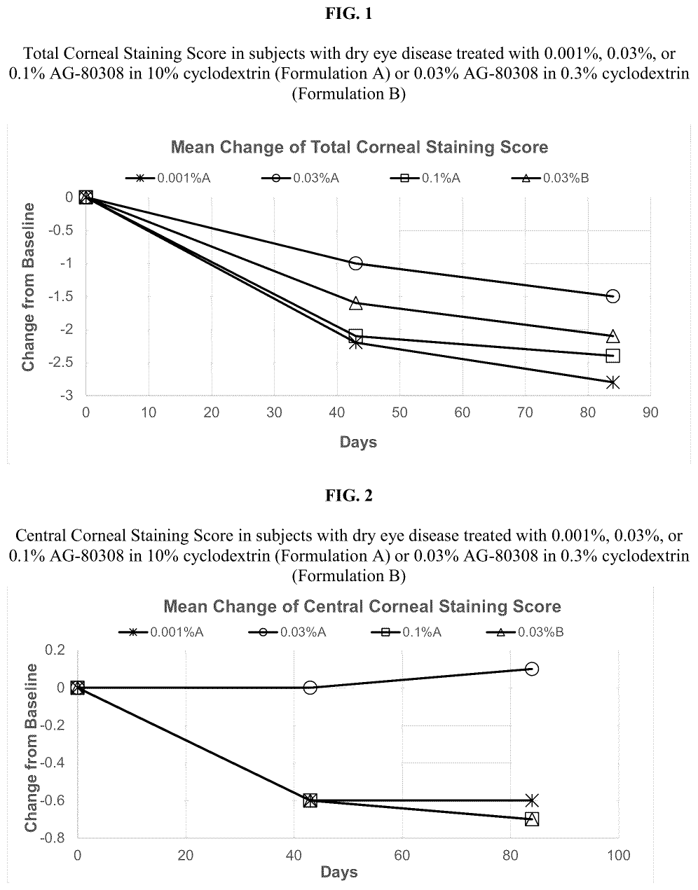

The results are shown in FIG. 1. AG-80308 Formulation A (0.001%) provided the greatest reduction of total corneal staining score.

### Central Corneal Staining Score

Central staining score at baseline (Day 1) was 1.8 [0.70], 1.3 [0.66], 1.8 [0.77] and 1.8 [0.75] in the group treated with Formulation A (0.001%), (0.03%), (0.1%) and Formulation B, respectively.

At Visit 4 (Day 43), all treatment groups showed improvement in the mean change from baseline. Changes from baseline were −0.5 [0.17], −0.4 [0.21], −0.5 [0.18] and −0.4 [0.18] in the group treated with Formulation A (0.001%), (0.03%), (0.1%) and Formulation B, respectively.

At Visit 5 (Day 84), similar improvements were observed. Changes from baseline were −0.6 [0.16], −0.3 [0.20], −0.6 [0.17], and −0.5 [0.17] in the group treated with Formulation A (0.001%), (0.03%), (0.1%) and Formulation B, respectively.

The results are shown in FIG. 2. Formulations A (0.001%) and (0.1%) and Formulation B provided the greatest reduction of central corneal staining score.

### 7-Item VAS Assessment (Symptom Relief)

The VAS symptom assessment was performed at Visit 1 (Day −30 to Day −2), Visit 2 (Day 1), Visit 3 (Day 15), Visit 4 (Day 43), and Visit 5 (Day 84). Subjects were asked to rate each ocular symptom, unrelated to study drug instillation, by placing a vertical mark on the horizontal line to indicate the level of discomfort. Zero millimeters (mm) corresponds to “no discomfort” and 100 mm corresponds to “maximal discomfort. The symptoms evaluated were burning/stinging, itching, foreign body sensation, eye discomfort, eye dryness, photophobia, and pain. Subjects were instructed to evaluate their symptoms “at that moment” for both eyes.

### Eye Discomfort Score

Eye discomfort score at baseline (Day 1) was 62.7 [16.83], 59.6 [17.17], 56.2 [15.07] and 57.4 [14.06] in the groups treated with Formulation A (0.001%), (0.03%) and (0.1%) and Formulation B, respectively.

At Visit 3 (Day 15), Formulation A (0.001%) and Formulation B (0.03%) treatment groups showed improvement in the mean change from baseline.

At Visit 4 (Day 43), similar improvements were observed. The Formulation A 0.03% treatment group showed no improvement from baseline. Changes from baseline were −9.0 [5.54]), 6.0 [6.29], −4.4 [5.59] and −20.0 [5.57] in the groups treated with Formulation A (0.001%), (0.03%) and (0.1%) and Formulation B, respectively.

At Visit 5 (Day 84), all treatment groups showed improvement in the mean change from baseline. Changes from baseline were −13.9 [6.10], −4.0 [6.92], −10.5 [6.37] and −28.8 [6.29] in the groups treated with Formulation A (0.001%), (0.03%) and (0.1%) and Formulation B, respectively.

The results are shown in FIG. 3. Formulation B provided the greatest reduction of eye discomfort score starting from day 15.

### Eye Dryness Score

Eye dryness score at baseline (Day 1) was 61.4 [11.19], 63.9 [11.32], 63.6 [12.40] and 62.8 [10.87] in the groups treated with Formulation A (0.001%), (0.03%) and (0.1%) and Formulation B, respectively.

At Visit 3 (Day 15), the groups treated with Formulation A (0.1%) and Formulation B showed the greatest improvement in the mean change from baseline. Changes from baseline were −0.4 [4.31], −0.9 [4.82], −10.5 [4.54] and −16.2 [4.42] in the groups treated with Formulation A (0.001%), (0.03%) and (0.1%) and Formulation B, respectively.

At Visit 4 (Day 43), groups treated with Formulation A (0.001%), (0.03%) and (0.1%) and Formulation B showed improvement in the mean change from baseline. 0.03% A treatment group showed no improvement from baseline. Changes from baseline were −8.2 [5.19], 6.5 [6.05], −10.0 [5.33], and −15.1 [5.33] in groups treated with Formulation A (0.001%), (0.03%) and (0.1%) and Formulation B, respectively.

At Visit 5 (Day 84), all treatment groups showed improvement in the mean change from baseline. Changes from baseline were −12.8 [5.78], −3.4 [6.74], −8.6 [6.11] and −23.2 [6.11] in groups treated with Formulation A (0.001%), (0.03%) and (0.1%) and Formulation B, respectively.

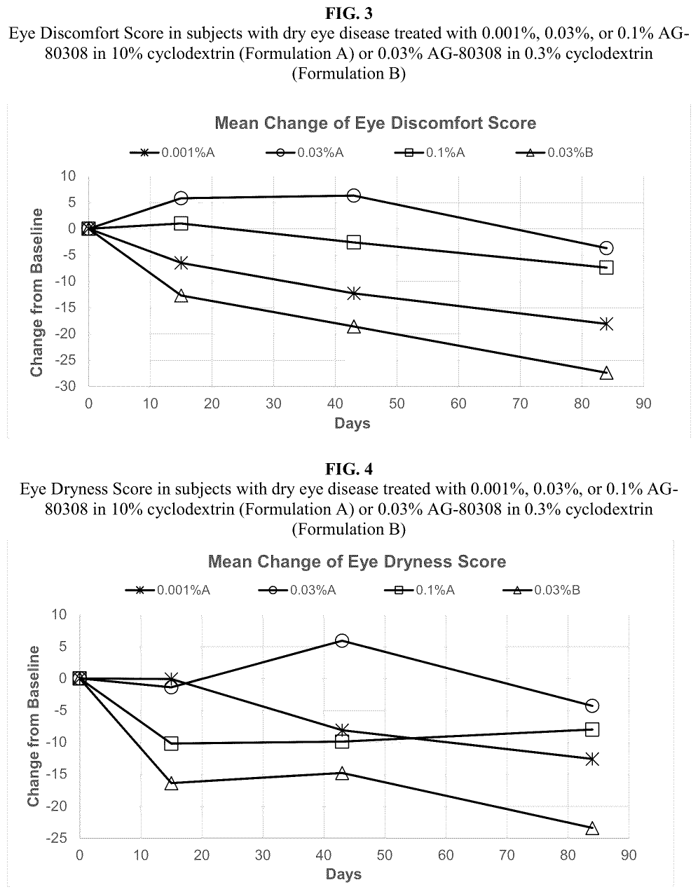

The results are shown in FIG. 4. 0.03% in Formulation B provided the greatest reduction of eye dryness score starting from day 15.

AG-80308 showed clinically meaningful unit reductions in corneal and conjunctival staining, 7-item visual analog scale for dry eye symptoms, and ocular surface disease index scores. Corneal staining was significantly improved from baseline by Day 43 (p<0.05) and Day 84 (p<0.05) with unit improvements from baseline of −2.1±0.57 and −2.7±0.57 (LS Mean±SE), respectively. For eye dryness and discomfort, Formulation B 0.03% provided the greatest reduction of symptoms from baseline to Day 15 (p<0.02), Day 43 (p<0.02) and Day 84 (p<0.002) with unit improvements from baseline of −14.1±4.09, −20.0±5.57, and −28.8±6.29, respectively.

AG-80308 was proven safe when given BID for 3 months in both formulations. There was a dose-dependent increase in treatment emergent adverse events (TEAEs) with Formulation A which were ocular in nature and consisted of mild to moderate discharge and eyelid margin crusting. The TEAEs are believed to be related to the pharmacological activity of AG-80308. The safety profile of AG-80308 was markedly improved with Formulation B compared to that of Formulation A.

What is claimed is:

1. A pharmaceutical composition comprising from 0.001% (w/v) to 10% (w/v) AG-80308 and from 0.1% to 10% (w/v) cyclodextrin.

2. The pharmaceutical composition of claim 1 comprising from 0.001% (w/v) to 0.04% (w/v) AG-80308 and from 0.01% to 0.4% (w/v) cyclodextrin.

3. The pharmaceutical composition of claim 1 comprising 0.001% (w/v), 0.002% (w/v), 0.003% (w/v), 0.005% (w/v), 0.01% (w/v), 0.02% (w/v), or 0.03% (w/v) AG-80308 and 0.01% (w/v), 0.02% (w/v), 0.03%, (w/v) 0.05% (w/v), 0.1% (w/v), 0.2% (w/v), or 0.3% (w/v) cyclodextrin.

4. The pharmaceutical composition of claim 1 comprising AG-80308 and cyclodextrin in the amounts selected from the group consisting of: 0.002% (w/v) AG-80308 and 0.02% (w/v) cyclodextrin; 0.002% (w/v) AG-80308 and 0.2% (w/v) cyclodextrin; 0.006% (w/v) AG-80308 and 0.06% (w/v) cyclodextrin; 0.006% (w/v) AG-80308 and 0.2% (w/v) cyclodextrin; 0.02% (w/v) AG-80308 and 0.2% (w/v) cyclodextrin; and 0.03% (w/v) AG-80308 and 0.3% (w/v) cyclodextrin.

5. The pharmaceutical composition of claim 1 formulated for topical ocular delivery.

6. The pharmaceutical composition of claim 5, wherein cyclodextrin is 2-hydroxypropyl-beta-cyclodextrin (HPβCD).

7. The pharmaceutical composition of claim 5 further comprising citric acid monohydrate and/or sodium phosphate dibasic heptahydrate as a buffer.

8. The pharmaceutical composition of claim 7, wherein the buffer is citric acid monohydrate at a concentration of from 0.01% to 1% (w/v), and/or sodium phosphate dibasic heptahydrate at a concentration of from of 0.01 to 1% (w/v).

9. The pharmaceutical composition of claim 5 with a pH of between 4.5 and 8.0.

10. A method for treating an ocular inflammatory disease in a subject in need thereof comprising administering to said subject a pharmaceutical composition comprising from 0.001% (w/v) to 10% (w/v) AG 80308 and from 0.1% to 10% (w/v) cyclodextrin.

11. The method of claim 10, wherein the pharmaceutical composition is formulated for topical ocular delivery.

12. The method of claim 11, wherein the pharmaceutical composition comprises from 0.001% (w/v) to 0.04% (w/v) AG-80308 and from 0.01% to 0.4% (w/v) cyclodextrin.

13. The method of claim 11, wherein the pharmaceutical composition comprises 0.001% (w/v), 0.002% (w/v), 0.003% (w/v), 0.005% (w/v), 0.01% (w/v), 0.02% (w/v), or 0.03% (w/v) AG-80308 and 0.01% (w/v), 0.02% (w/v), 0.03%, (w/v) 0.05% (w/v), 0.1% (w/v), 0.2% (w/v), or 0.3% (w/v) cyclodextrin.

14. The method of claim 11, wherein the pharmaceutical composition comprises AG-80308 and cyclodextrin in the amounts selected from the group consisting of: 0.002% (w/v) AG-80308 and 0.02% (w/v) cyclodextrin; 0.002% (w/v) AG-80308 and 0.2% (w/v) cyclodextrin; 0.006% (w/v) AG-80308 and 0.06% (w/v) cyclodextrin; 0.006% (w/v) AG-80308 and 0.2% (w/v) cyclodextrin; 0.02% (w/v) AG-80308 and 0.2% (w/v) cyclodextrin; and 0.03% (w/v) AG-80308 and 0.3% (w/v) cyclodextrin.

15. The method of claim 11, wherein cyclodextrin is 2-hydroxypropyl-beta-cyclodextrin (HPβCD).

16. The method of claim 11, wherein the pharmaceutical composition further comprises citric acid monohydrate and/or sodium phosphate dibasic heptahydrate as a buffer.

17. The method of claim 16, wherein the buffer is citric acid monohydrate at a concentration of from 0.01% to 1% (w/v), and/or sodium phosphate dibasic heptahydrate at a concentration of from 0.01 to 1% (w/v).

18. The method of claim 11, wherein one drop of said pharmaceutical composition is administered per eye 1 to 6 times a day.

19. The method of claim 18, wherein one drop of said pharmaceutical composition is administered per eye twice a day.

20. The method of claim 10, wherein said ocular inflammatory disease is dry eye disease, keratitis, uveitis, conjunctivitis, ocular graft-vs.-host disease (GvHD), allergic eye disease, post-surgical corneal inflammation, blepharitis, MGD, or Sjogren's syndrome.

21. The method of claim 11, wherein said pharmaceutical composition is administered in combination with one or more selected from the group consisting of a steroid and a non-steroidal anti-inflammatory drug.

22. The method of claim 11, wherein said pharmaceutical composition has a pH of between 4.5 and 8.0.
